(19) United States
(12) Patent Application Publication
(10) Pub. No.: US 2021/0290181 A1
(43) Pub. Date: Sep. 23, 2021

(54) SYSTEM AND METHOD FOR MONITORING, IDENTIFYING AND REPORTING IMPACT EVENTS IN REAL-TIME

(71) Applicant: ANIRUDHA SURABHI VENKATA JAGANNADHA RAO, Dallas, TX (US)

(72) Inventor: ANIRUDHA SURABHI VENKATA JAGANNADHA RAO, Dallas, TX (US)

(21) Appl. No.: 17/201,112

(22) Filed: Mar. 15, 2021

Related U.S. Application Data
(60) Provisional application No. 62/990,456, filed on Mar. 17, 2020.

(51) Int. Cl. A61B 5/00; A61B 5/11

(52) U.S. Cl. CPC A61B 5/7282; A61B 5/1114; A61B 5/1121; A61B 5/4064; A61B 2560/0475; A61B 5/1112; A61B 5/1128; A61B 2503/10; A61B 2562/0219; A61B 5/0004

(57) ABSTRACT

Exemplary embodiment of the present disclosure directed towards system for monitoring, identifying and reporting impact events in real-time, comprising: an impact event monitoring device monitors impact events of objects and subjects through a processing device. The processing device identifies accurate positions and locations and sensor data of objects; and subjects; the processing device enables an image capturing unit to capture and record objects; and subjects, a network module reports accurate positions and locations and sensor data, media files of impact events to a first computing device; a second computing device; and an impact event reporting module enables the first computing device and the second computing device to analyze accurate positions and locations of objects; subjects; to understand the extent of the impact events.

### CROSS-REFERENCE TO RELATED APPLICATIONS

This patent application claims priority benefit of U.S. Provisional Patent Application No. 62990456, entitled “System And Method For Monitoring, Identifying And Reporting Impact Events In Real-Time”, filed on 17-Mar.-2020. The entire contents of the patent application is hereby incorporated by reference herein in its entirety.

### COPYRIGHT AND TRADEMARK NOTICE

This application includes material which is subject or may be subject to copyright and/or trademark protection. The copyright and trademark owner(s) has no objection to the facsimile reproduction by any of the patent disclosure, as it appears in the Patent and Trademark Office files or records, but otherwise reserves all copyright and trademark rights whatsoever.

### TECHNICAL FIELD

The disclosed subject matter relates generally to an emergency event management system. More particularly, the present disclosure relates to a system and method for monitoring, identifying and reporting impact events that occur to objects/subjects in real-time to an end-user.

### BACKGROUND

Generally, the participation of athletes in athletic activities is increasing at all age levels. All participants are potentially exposed to physical harm as a result of such participation. The physical harm is more likely to occur in athletic events where collisions between participants frequently occur (e.g., football, field hockey, lacrosse, ice hockey, soccer and so forth). In connection with sports such as football, hockey, and lacrosse where deliberate collisions between participants occur, the potential for physical harm and/or injury is greatly enhanced.

For example, in the world each year there are a million athletes with an age below twenty-four who play contact sports such as football, basketball, hockey, soccer, boxing and mixed martial arts (mixed martial arts (MMA)). All these young athletes are at risk for head injury with a concussion (concussive traumatic brain injuries (CTBI)) and long-term brain dysfunction due to repeated head impacts. These young athletes with developing neurological systems, suffer a large part of the 3 8 million CTBI that occur annually and are at high risk of developing long-term adverse neurological, physiological and cognitive deficits. The conditions of head impacts responsible for CTBI and potential long-term deficits in athletes are unknown. Head injuries are caused by positive and negative acceleration forces experienced by the brain and may result from linear or rotational accelerations (or both). Both linear and rotational accelerations are likely to be encountered by the head at impact, damaging neural and vascular elements of the brain. Similarly, the percentage of vehicular crashes both on-road and off-road has been increasing rapidly all over the world. In most cases, the information on the state of the vehicle during the incident is not known. Many of the deaths and permanent injuries could have been prevented if the emergency responders would have arrived more quickly. Too many precious minutes are lost because calls for help are delayed, or because emergency responders cannot quickly locate the accident. Hence, there is a need for a system to sense the impact events and activate the emergency protocol examiners to properly diagnose the extent and severity of the injury.

In the light of the aforementioned discussion, there exists a need for a system with novel methodologies that would overcome the above-mentioned challenges.

### SUMMARY

The following presents a simplified summary of the disclosure in order to provide a basic understanding of the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

Exemplary embodiments of the present disclosure are directed towards a system and method for monitoring, identifying and reporting the impact events that occur to objects/subjects in real-time to the end-user.

An objective of the present disclosure is directed towards providing a wirelessly linked impact, anomaly sensing, and reporting system.

Another objective of the present disclosure is directed towards activating an emergency protocol automatically by an impact event reporting module.

Another objective of the present disclosure is directed towards delivering additional data to medical examiners to properly diagnose the extent and severity of the injury.

Another objective of the present disclosure is directed towards identifying the accurate positions of head and/or body position of the subject and the geographical location of the object/subject at the time of the impact event and are analyzed by medical professionals to gauge the extent of the injury.

According to an exemplary aspect, an impact event monitoring device configured to monitor one or more impact events of at least one of: objects; and subjects; through a processing device.

According to another exemplary aspect, the processing device configured to enable an image capturing unit to capture and record at least one of: the objects; and the subjects. The processing device configured to identify one or more accurate positions and locations and sensor data of at least one of: the objects; and the subjects;

According to another exemplary aspect, a network module configured to report the one or more accurate positions and locations and the sensor data, one or more media files of the one or more impact events to at least one of: a first computing device; a second computing device.

According to another exemplary aspect, an impact event reporting module configured to enable the at least one: the first computing device; and the second computing device; to analyze the one or more accurate positions and locations, the sensor data, and the one or more media files of at least one of: the objects; the subjects; to understand the extent of the one or more impact events.

### BRIEF DESCRIPTION OF THE DRAWINGS

FIG. 1 is a block diagram depicting a schematic representation of a system for monitoring, identifying and reporting impact events occur to objects in real-time.

FIG. 2 is a block diagram depicting an impact event monitoring device 102 shown in FIG. 1, in accordance with one or more exemplary embodiments.

FIG. 3 is a block diagram depicting a schematic representation of the impact event reporting module 114 shown in FIG. 1, in accordance with one or more exemplary embodiments.

FIG. 4 is a flowchart depicting an exemplary method of reporting impact events to the second end users, in accordance with one or more exemplary embodiments.

FIG. 5 is a flowchart depicting an exemplary method of tracking the objects using an impact event monitoring device, in accordance with one or more exemplary embodiments.

FIG. 6 is a flowchart depicting an exemplary method of displaying the activity recognition and performance grading of the objects, in accordance with one or more exemplary embodiments.

FIG. 7 is a flowchart depicting an exemplary method of displaying the movements of objects/subjects during the crash, in accordance with one or more exemplary embodiments.

FIG. 8 is a block diagram illustrating the details of a digital processing system 800 in which various aspects of the present disclosure are operative by execution of appropriate software instructions.

### DETAILED DESCRIPTION OF EXAMPLE EMBODIMENTS

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced items. Further, the use of terms “first”, “second”, and “third”, and so forth, herein do not denote any order, quantity, or importance, but rather are used to distinguish one element from another.

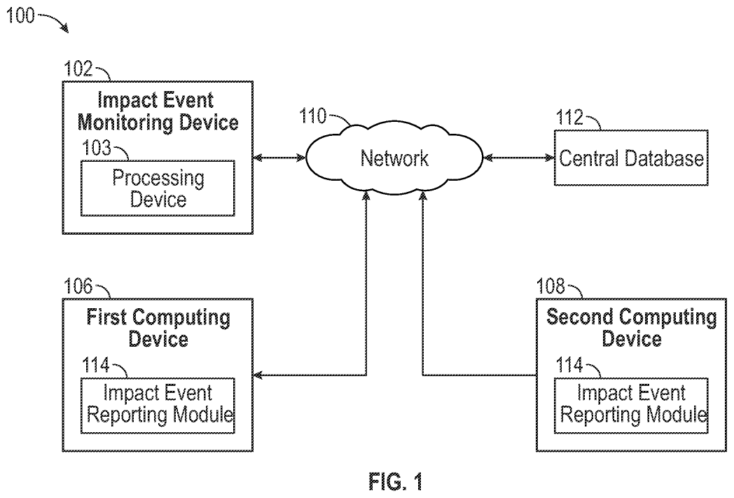

Referring to FIG. 1 is a block diagram 100 depicting a schematic representation of a system for monitoring, identifying and reporting impact events that occur to objects in real-time, in accordance with one or more exemplary embodiments. The impact events may include, but not limited to, non-accidental emergency events relating to the vehicle (e.g., a theft of the vehicle), or emergency events relating specifically to the occupant(s) of the vehicle (e.g., a medical impairment of an occupant of the vehicle, regular events in the course of rough activity, a kidnapping or assault of an occupant of the vehicle, etc.), accidental emergency events relating to vehicle or other transport crashes, fires, medical emergencies, or other threats to safety, movements and motion, injury, abnormalities, and so forth. The system 100 includes an impact event monitoring device 102, a processing device 103, a first computing device 106, a second computing device 108, a network 110, a central database 112 and an impact event reporting module 114. The system 100 may include multiple impact event monitoring devices 102, multiple processing devices 103, and multiple computing devices 106, 108. The system 100 may link multiple impact event monitoring devices 102, multiple processing devices 103, and multiple computing devices 106, 108 into a single hub that may display devices information at a glance.

The impact event monitoring device 102 may be an inertial measurement unit. The impact event monitoring device 102 may be configured to detect and track an object's motion in three-dimensional space, and allows the first end users to interact with the first computing device 106 by tracking motion in free space and delivering these motions as input commands The impact event monitoring device 102 may be integrated into a vehicle, steering wheel, dashboard, car seats (if the user does not require an image capturing unit), headbands, helmets, electronic device, and so forth. The impact event monitoring device 102 may be configured to detect/sense the impact events, emergency events, interrupts, impacts or anomalies occur to the objects/subjects. The impact event monitoring device 102 may be configured to activate the impact protocol (emergency protocol) to establish the communication with the first computing device 106 and the second computing device 108 through the impact event reporting module 114 via the network 110. The objects may include, but not limited to, vehicles, car seats, wristbands, helmets, headbands, and so forth. The subject may be a first end user. The first end user may include, but not limited to, a driver, an athlete, a motorist, passenger, a vehicle owner, a vehicle user, an individual, and so forth. The network 110 may include but not limited to, an Internet of things (IoT network devices), an Ethernet, a wireless local area network (WLAN), or a wide area network (WAN), a Bluetooth low energy network, a ZigBee network, a WIFI communication network e.g., the wireless high speed internet, or a combination of networks, a cellular service such as a 4G (e.g., LTE, mobile WiMAX) or 5G cellular data service, a RFID module, a NFC module, wired cables, such as the world-wide-web based Internet, or other types of networks may include Transport Control Protocol/Internet Protocol (TCP/IP) or device addresses (e.g. network-based MAC addresses, or those provided in a proprietary networking protocol, such as Modbus TCP, or by using appropriate data feeds to obtain data from various web services, including retrieving XML data from an HTTP address, then traversing the XML for a particular node) and so forth without limiting the scope of the present disclosure. The impact event reporting module 114 may be configured to establish the communication between the impact event monitoring device 102 and the first computing device 104 through the network 110.

The first computing device 106 and the second computing device 108 may be operatively coupled to each other through the network 110. The first and second computing devices 106 and 108 may include but not limited to, a computer workstation, an interactive kiosk, and a personal mobile computing device such as a digital assistant, a mobile phone, a laptop, and storage devices, backend servers hosting the database and other software, and so forth. The first computing device 106 may be operated by the first end user. The second computing device 108 may be operated by the second end user. The second end user may include, but not limited to, medical professionals, a medical examiner(s), an emergency responder(s), an emergency authority medical practitioner(s), a doctor(s), a physician(s), a family member(s), a friend(s), a relative(s), a neighbour(s), an emergency service provider(s), and so forth.

Although the first and second computing devices 106, 108 are shown in FIG. 1, an embodiment of the system 100 may support any number of computing devices. Each computing device supported by the system 100 is realized as a computer-implemented or computer-based device having the hardware or firmware, software, and/or processing logic needed to carry out the intelligent messaging techniques and computer-implemented methodologies described in more detail herein.

The impact event reporting module 114, which is accessed as mobile applications, web applications, software that offers the functionality of accessing mobile applications, and viewing/processing of interactive pages, for example, are implemented in the first and second computing devices 106, 108 as will be apparent to one skilled in the relevant arts by reading the disclosure provided herein. The impact event reporting module 114 may be downloaded from the cloud server (not shown). For example, the impact event reporting module 114 may be any suitable applications downloaded from, GOOGLE PLAY® (for Google Android devices), Apple Inc.'s APP STORE® (for Apple devices, or any other suitable database). In some embodiments, the impact event reporting module 114 may be software, firmware, or hardware that is integrated into the first and second computing devices 106, 108.

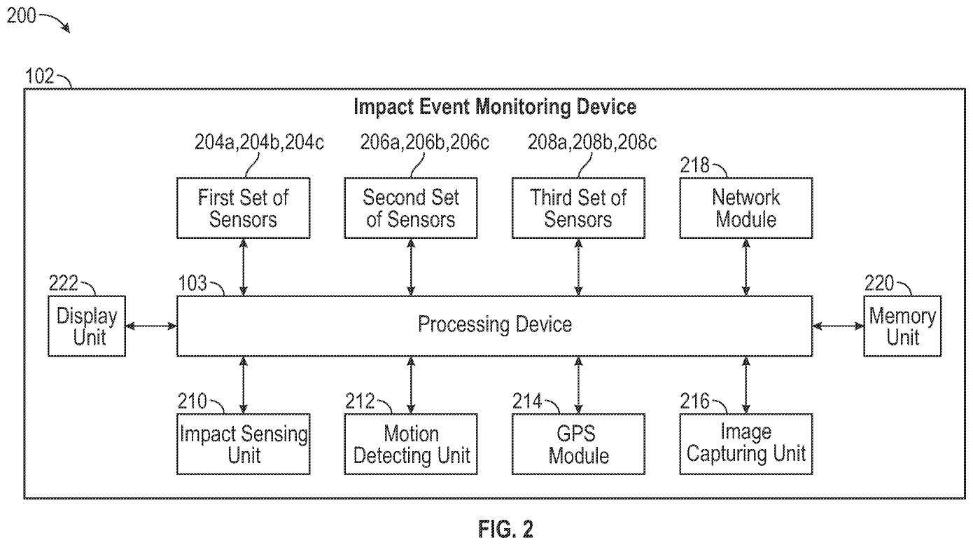

The processing device 103 may include, but not limited to, a microcontroller (for example ARM 7 or ARM 11), a raspberry pi3 or a Pine 64or any other 64 bit processor which can run Linux OS, a microprocessor, a digital signal processor, a microcomputer, a field programmable gate array, a programmable logic device, a state machine or logic circuitry, Arduino board. A set of sensors (204a, 204band 204c, 206a, 206b and 206c, 208a, 208b and 208c shown in FIG. 2) may be electrically coupled to the processing device 102.

According to exemplary embodiment of the present disclosure, a system for monitoring, identifying and reporting impact events in real-time, includes the impact event monitoring device configured to monitor impact events of objects and subjects through the processing device 102, the processing device 102 configured to identify one or more accurate positions and locations, and sensor data of the objects and the subjects. The accurate positions and locations may include, but not limited to, a head and/or body position of the subject, a geographical position of the object, a geographical position of the subject and so forth. The sensor data may include, but not limited to, quaternions, Euler angles, vital statistics, the rotational angle of the head of the individual or the object, movement and/or motion of the individual or the object, location acceleration and gyroscope vectors, velocity, location and so forth. The processing device 102 configured to enable the image capturing unit 216 (as shown in FIG. 2) to capture and record the objects and the subjects.

The network module 218 (as shown in FIG. 2) configured to report the accurate positions and locations, the sensor data, media files of the impact events to the first computing device 106 and the second computing device 108. The media files may include, but not limited to, images, pictures, videos, GIF's, and so forth. The impact event reporting module 114 configured to enable the first computing device 106 and the second computing device 108 to analyze the accurate positions and locations of the objects and the subjects to understand the extent of the impact events.

According to exemplary embodiment of the present disclosure, the system for monitoring, identifying and reporting impact events in real-time, comprising an impact event monitoring device 102 is configured to monitor impact events of objects; and subjects, the impact event monitoring device 102 is configured to identify the accurate positions and locations of the objects and the subjects and activates the impact protocol to establish communication with the first computing device 106 and the second computing device 108 over the network 110, the impact event monitoring device 102 is configured to deliver notifications of the impact events of the objects and subjects to the second computing device 108 over the network 110.

A method for monitoring, identifying and reporting impact events in real-time, comprising: monitoring objects and subjects by the event monitoring device 102; detecting accurate positions and locations and sensor data by the impact event monitoring device 102; capturing and recording the objects and the subjects and establishing communication between the impact event monitoring device 102 with the first computing device 106 and the second computing device 108 through the network module 218; reporting accurate positions and locations; the sensor data; media files; from the impact event monitoring device 102 to the first computing device 106 and the second computing device 108 and analyzing the accurate positions and locations; the sensor data; the media files of the objects by the impact event reporting module 114 for understanding the extent and severity of the impact events.

Referring to FIG. 2 is a block diagram 200 depicting the impact event monitoring device 102 shown in FIG. 1, in accordance with one or more exemplary embodiments. The impact event monitoring device 102 includes the processing device 203, a first set of sensors 204a, 204b and 204c, a second set of sensors 206a, 206b and 206c, a third set of sensors 208a, 208b, and 208c, an impact sensing unit 210, a motion detecting unit 212, a GPS module 214, an image capturing unit 216, and a network module 218, a memory unit 220, and a display unit 222. The first set of sensors 204a, 204b, 204c, the second set of sensors 206a, 206b and 206c, the third set of sensors 208a, 208b, and 208c may include, but not limited to, gyroscopes, accelerometers, compasses, pressure sensors, and magnetometers.

The first set of sensors 204a, 204b and 204c may be electrically coupled to the processing device 203 and is configured to measure the linear acceleration and/or angular acceleration of the sensor array. The second set of sensors 206a, 206b and 206c may be electrically coupled to the processing device 203 and is configured to calibrate the exact orientations by measuring the Euler angles and/or quaternions. The third set of sensors 208a, 208b and 208c may be electrically coupled to the processing device 203 and is configured to monitor vital statistics, the rotational angle of the head of the individual or the object at the time of the impact event. The third set of sensors 208a, 208b and 208c may also be configured to provide additional data to the second end users to properly diagnose the extent and severity of the impact event. The impact sensing unit 210 may be electrically coupled to the processing device 203 and is configured to detect and determine the impact events that occur to the objects/subjects. The motion detecting unit 212 may be electrically coupled to the processing device 203 and is configured to measure changes in the orientations for having a continuous replication of the movement and/or motion of the objects/subjects.

The GPS module 214 may be electrically coupled to the processing device 203 and is configured to detect the accurate location of the impact events that occur to the objects/subjects. The image capturing unit 216 may be electrically coupled to the processing device 203 and is configured to record the video of the subjects/objects and capture the objects/subjects. For example, similar to live media, in the sense the image capturing unit 216 starts recording as soon as the first end user opens the impact event reporting module 114 before the live media is captured. The live media may include, but not limited to, live photos, live media files, and so forth. The image capturing unit 216 may be configured to recreate the captured impact events (live media) in a 3D space. The network module 218 may be electrically coupled to the processing device 203 and is configured to connect the impact event monitoring device 102 with the first computing device 104. The network module 218 may be configured to send the impact events as impact notifications to the second end users. The impact notifications may include but not limited to, SMS, alerts, email, warnings, and so forth. The network module 218 may also be configured to send a geographical location as a communication link and the information identifying the location of the objects\subjects to the second computing device 108 to communicate the portion of data stored in the memory unit 220. The information stored in the memory unit 220 may be preserved at least until an acknowledgment of receipt is received representing successful transmission through the communication link. The memory unit 220 may be electrically coupled to the processing device 203 and is configured to receive movement or motion output and stores at least a portion of motion commencing at and/or before said determination. The display unit 222 may be electrically coupled to the processing device 203 and is configured to display the sensor data, impact notifications, and so forth.

According to exemplary embodiment of the present disclosure, the impact event monitoring device 102 includes the first set of sensors 204a, 204b, and 204c configured to measure the linear acceleration and the angular acceleration of the sensor array. The second set of sensors 206a, 206b, and 206c are configured to calibrate orientations of the objects and the subjects by measuring Euler angles and/or quaternions. The third set of sensors 208a, 208b, and 208c are configured to monitor the vital statistics, rotational angle of a head of the subject at the time of the impact event. The third set of sensors 208a, 208b, and 208c are configured to provide an additional sensor data to the second computing device 108 for proper diagnosing extent and severity of the one or more impact events.

The impact sensing unit 210 is configured to detect and determine the impact events that occurs to the objects and the subjects. The motion detecting unit 212 is configured to measure changes in the orientations for continuous replication of a movement and/or motion of the objects and the subjects. The GPS module 214 is configured to detect the accurate location of the impact events that occurs to the objects and the subjects. The network module 218 is configured to establish communication between the impact event monitoring device 102 and the first computing device 106, the second computing device 108 to deliver notifications of the impact events. The network module 218 is configured to send the communication link with the remote location, and the information identifying the location of the objects and the subjects to the second computing device 108 for communicating the sensor data stored in the memory unit 220 of the impact event monitoring device 102.

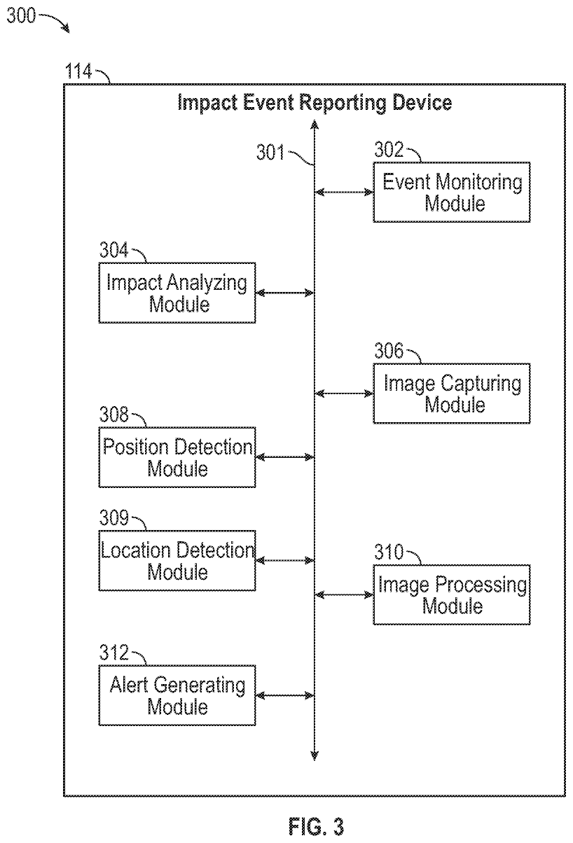

Referring to FIG. 3 is a block diagram 300 depicting a schematic representation of the impact event reporting module 114 shown in FIG. 1, in accordance with one or more exemplary embodiments. The impact event reporting module 114 includes a bus 301, an event monitoring module 302, an impact analyzing module 304, an image capturing module 306, a position detection module 308, a location detection module 309, and an image processing module 310, and an alert generating module 312. The bus 301 may include a path that permits communication among the modules of the impact event reporting module 114 installed on the computing device 106, 108. The term “module” is used broadly herein and refers generally to a program resident in the memory of the computing device 106, 108. The impact event reporting module 114 may include machine learning techniques and computer-implemented pattern recognition techniques to detect anomalies or variations in normal behavior.

The event monitoring module 302 may be configured to read the sensor data of the objects/subjects and stores in the central database 112. The sensor data may be measured by the impact event monitoring device 102. The sensor data may include, but not limited to, quaternions, Euler angles, vital statistics, the rotational angle of the head of the individual or the object, movement and/or motion of the individual or the object, geo location, acceleration and gyroscope vectors, velocity, location and so forth. The impact analyzing module 304 may be configured to analyze the impact events received from the processing device 103/203. The image capturing module 306 may be configured to capture the objects/subjects. The image capturing module 306 may be configured to move the media files of the impact event front and back with respect to time so that the second end-user may see the impact event from any angle. The media files may include, but not limited to, images, pictures, videos, GIF's, and so forth. The media files may be moved front and back with respect to time and the object/subject may be portrayed accordingly.

The position detection module 308 may be configured to fetch the object/subject positions “x” seconds before the impact interrupt and “x” seconds after the impact interrupt. The image processing module 310 may be configured to convert the resulting “2x” seconds of the object/subject positions into a short animation/video by which the accurate object/subject positions at the time of the impact event may be reproduced. The alert generating module 312 may be configured to generate the impact notifications to the second computing device 106 to estimate the extent of the impact event by the second end user. The impact notifications may be analysed by the second end user to understand the severity of the impact event.

According to exemplary embodiment of the present disclosure, the impact event reporting module includes the event monitoring module 302 is configured to read the sensor data of the objects and the subjects. The impact analyzing module 304 is configured to analyze impact events received from the processing device 102. The image capturing module 306 is configured to move the media files of the impact events front and back with respect to time and the object/subject may be portrayed accordingly. The position detection module 308 is configured to fetch a head and/or body position of the subjects before the impact events and after the impact events. The location detection module 309 is configured to fetch the geographical location (geo location) of the objects and/or subjects, before the impact events and after the impact events. The alert generating module 312 is configured to deliver the notifications of the impact events to the second computing device 108 for estimating the extent of the impact events by the second end user.

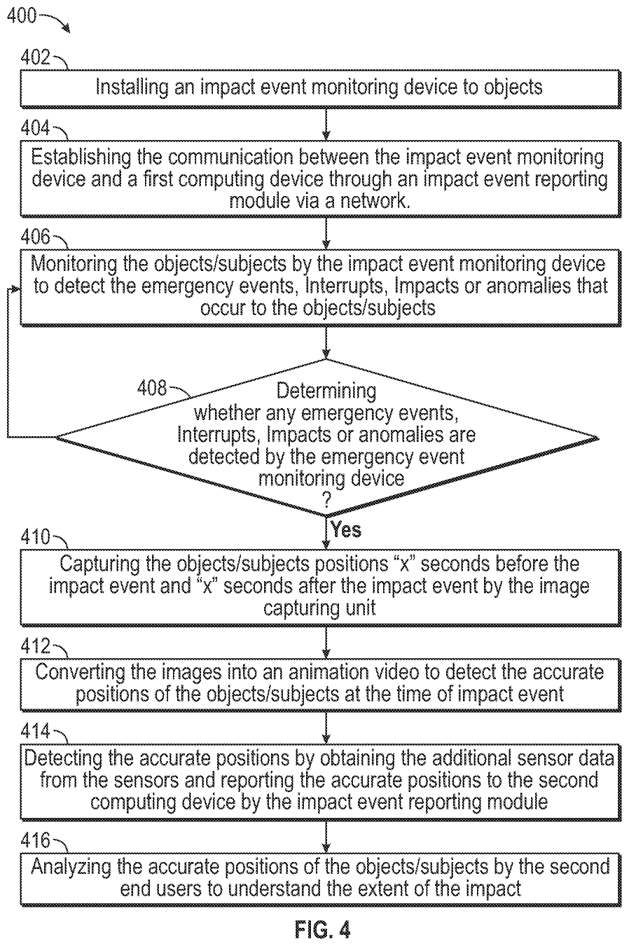

Referring to FIG. 4 is a flowchart 400 depicting an exemplary method of reporting impact events to the second end users, in accordance with one or more exemplary embodiments. As an option, the exemplary method 400 is carried out in the context of the details of FIG. 1, FIG. 2 and FIG. 3. However, the exemplary method 400 is carried out in any desired environment. Further, the aforementioned definitions are equally applied to the description below.

The method commences at step 402, installing the impact event monitoring device to objects. Thereafter at step 404, establishing the communication between the impact event monitoring device and the first computing device through the impact event reporting module via the network. Thereafter at step 406, monitoring the objects/subjects by the impact event monitoring device to detect the impact events occur to the objects/subjects. Determining whether any impact events, interrupts, impacts or anomalies detected by the impact event monitoring device, at step 408. If the answer to step 408 is YES, the method continuous at step 410, capturing the objects/subjects positions “x” seconds before the impact event and “x” seconds after the impact event by the image capturing unit. Thereafter at step 412, converting the media files of objects/subjects into an animation video to detect the accurate positions and locations of the objects/subjects at the time of the impact event. Thereafter at step 414, detecting the accurate positions and locations by obtaining the additional sensor data from the sensors and reporting the accurate positions and locations to the second computing device by the impact event reporting module. Thereafter at step 416, analyzing the accurate positions and locations of the objects/subjects by the second end users to understand the extent of the impact. If the answer to step 408 is NO, the method redirects at step 406.

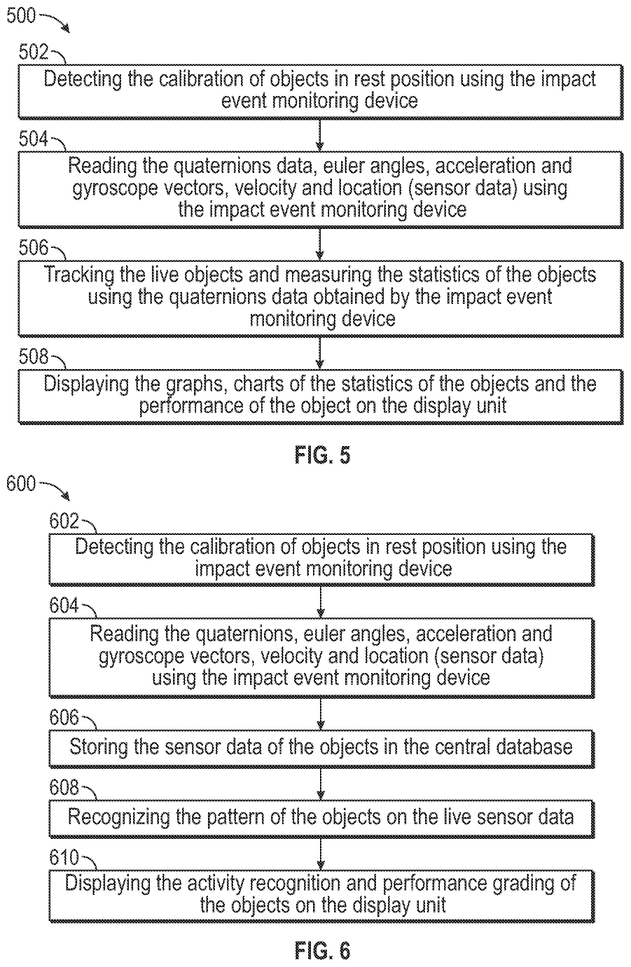

Referring to FIG. 5 is a flowchart 500 depicting an exemplary method of tracking the objects using the impact event monitoring device, in accordance with one or more exemplary embodiments. As an option, the exemplary method 500 is carried out in the context of the details of FIG. 1, FIG. 2, FIG. 3 and FIG.4. However, the exemplary method 500 is carried out in any desired environment. Further, the aforementioned definitions are equally applied to the description below.

The method commences at step 502, detecting the calibration of objects in rest position using the impact event monitoring device. Thereafter at step 504, reading the quaternions data, Euler angles, acceleration and gyroscope vectors, velocity and location (sensor data) using the impact event monitoring device. Thereafter at step 506, tracking the live objects and measuring the statistics of the objects using the quaternions data obtained by the impact event monitoring device. Thereafter at step 508, displaying the graphs, charts of the statistics of the objects and the performance of the object son the display unit.

Referring to FIG. 6 is a flowchart 600 depicting an exemplary method of displaying the activity recognition and performance grading of the objects, in accordance with one or more exemplary embodiments. As an option, the exemplary method 600 is carried out in the context of the details of FIG. 1, FIG. 2, FIG. 3, FIG.4, and FIG. 5. However, the exemplary method 600 is carried out in any desired environment. Further, the aforementioned definitions are equally applied to the description below.

The method commences at step 602, detecting the calibration of objects in rest position using the impact event monitoring device. Thereafter at step 604, reading the quaternions, Euler angles, acceleration and gyroscope vectors, velocity and location using the impact event monitoring device. Thereafter at step 606, storing the sensor data of the objects in the central database. Thereafter at step 608, recognizing the pattern of the objects on the live sensor data. Thereafter at step 610, displaying the activity recognition and performance grading of the objects on the display unit.

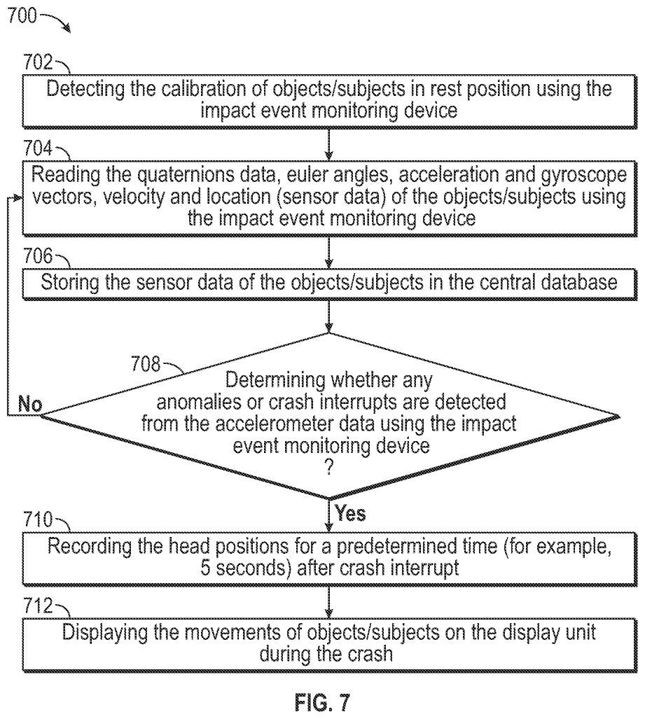

Referring to FIG. 7 is a flowchart 700 depicting an exemplary method of displaying the movements of objects/subjects during the crash, in accordance with one or more exemplary embodiments. As an option, the exemplary method 700 is carried out in the context of the details of FIG. 1, FIG. 2, FIG. 3 and FIG.4. However, the exemplary method 700 is carried out in any desired environment. Further, the aforementioned definitions are equally applied to the description below.

The method commences at step 702, detecting the calibration of objects/subjects in rest position using the impact event monitoring device. Thereafter at step 704, reading the quaternions data, Euler angles, acceleration and gyroscope vectors, velocity and location (sensor data) of the objects/subjects using the impact event monitoring device. Thereafter at step 706, storing the sensor data of the objects/subjects in the central database. Determining whether any anomalies or crash interrupts are detected from the accelerometer data using the impact event monitoring device, at step 708. If the answer to step 708 is YES, the method continues at step 710, recording the head positions for a predetermined time (Fox example, 5 seconds) after crash interrupt. Thereafter at step 712, displaying the movements of objects/subjects on the display unit during the crash. If the answer at step 708 is NO, the method redirects at step 704.

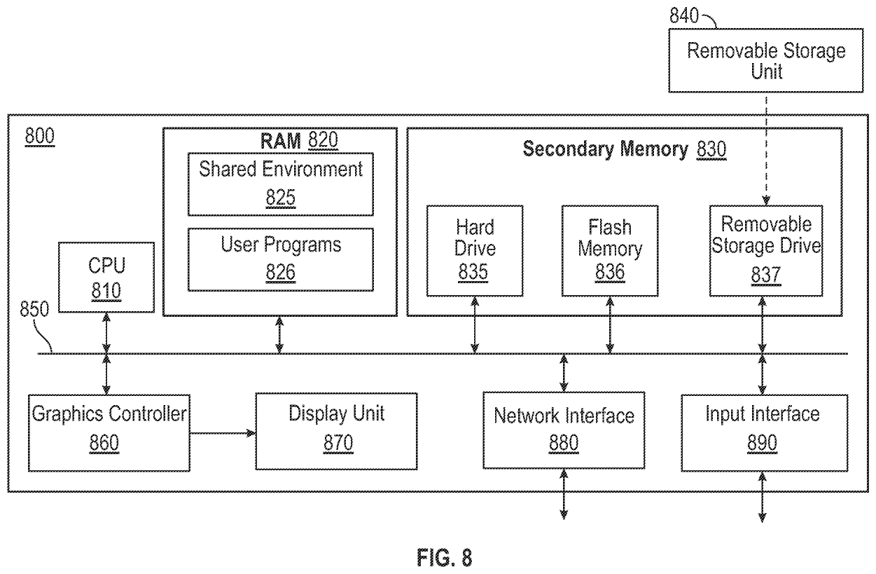

Referring to FIG. 8 is a block diagram 800 illustrating the details of a digital processing system 800 in which various aspects of the present disclosure are operative by execution of appropriate software instructions. The Digital processing system 800 may correspond to the computing devices 106, 108 (or any other system in which the various features disclosed above can be implemented).

Digital processing system 800 may contain one or more processors such as a central processing unit (CPU) 810, random access memory (RAM) 820, secondary memory 827, graphics controller 860, display unit 870, network interface 880, and input interface 890. All the components except display unit 870 may communicate with each other over communication path 850, which may contain several buses as is well known in the relevant arts. The components of FIG. 8 are described below in further detail.

CPU 810 may execute instructions stored in RAM 820 to provide several features of the present disclosure. CPU 810 may contain multiple processing units, with each processing unit potentially being designed for a specific task. Alternatively, CPU 810 may contain only a single general-purpose processing unit.

RAM 820 may receive instructions from secondary memory 830 using communication path 850. RAM 820 is shown currently containing software instructions, such as those used in threads and stacks, constituting shared environment 825 and/or user programs 826. Shared environment 825 includes operating systems, device drivers, virtual machines, etc., which provide a (common) run time environment for execution of user programs 826.

Graphics controller 860 generates display signals (e.g., in RGB format) to display unit 870 based on data/instructions received from CPU 810. Display unit 870 contains a display screen to display the images defined by the display signals. Input interface 890 may correspond to a keyboard and a pointing device (e.g., touch-pad, mouse) and may be used to provide inputs. Network interface 880 provides connectivity to a network (e.g., using Internet Protocol), and may be used to communicate with other systems (such as those shown in FIG. 1) connected to the network 106.

Secondary memory 830 may contain hard drive 835, flash memory 836, and removable storage drive 837. Secondary memory 830 may store the data software instructions (e.g., for performing the actions noted above with respect to the Figures), which enable digital processing system 800 to provide several features in accordance with the present disclosure.

Some or all of the data and instructions may be provided on removable storage unit 840, and the data and instructions may be read and provided by removable storage drive 837 to CPU 810. Floppy drive, magnetic tape drive, CD-ROM drive, DVD Drive, Flash memory, removable memory chip (PCMCIA Card, EEPROM) are examples of such removable storage drive 837.

Removable storage unit 840 may be implemented using medium and storage format compatible with removable storage drive 837 such that removable storage drive 837 can read the data and instructions. Thus, removable storage unit 840 includes a computer readable (storage) medium having stored therein computer software and/or data. However, the computer (or machine, in general) readable medium can be in other forms (e.g., non-removable, random access, etc.).

In this document, the term “computer program product” is used to generally refer to removable storage unit 840 or hard disk installed in hard drive 835. These computer program products are means for providing software to digital processing system 800. CPU 810 may retrieve the software instructions, and execute the instructions to provide various features of the present disclosure described above.

The term “storage media/medium” as used herein refers to any non-transitory media that store data and/or instructions that cause a machine to operate in a specific fashion. Such storage media may comprise non-volatile media and/or volatile media. Non-volatile media includes, for example, optical disks, magnetic disks, or solid-state drives, such as storage memory 830. Volatile media includes dynamic memory, such as RAM 820. Common forms of storage media include, for example, a floppy disk, a flexible disk, hard disk, solid-state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire and fiber optics, including the wires that comprise bus (communication path) 850. Transmission media can also take the form of acoustic or light waves, such as those generated during radio-wave and infra-red data communications.

Reference throughout this specification to “one embodiment”, “an embodiment”, or similar language means that a particular feature, structure, or characteristic described in connection with the embodiment is included in at least one embodiment of the present disclosure. Thus, appearances of the phrases “in one embodiment”, “in an embodiment” and similar language throughout this specification may, but do not necessarily, all refer to the same embodiment.

Furthermore, the described features, structures, or characteristics of the disclosure may be combined in any suitable manner in one or more embodiments. In the above description, numerous specific details are provided such as examples of programming, software modules, user selections, network transactions, database queries, database structures, hardware modules, hardware circuits, hardware chips, etc., to provide a thorough understanding of embodiments of the disclosure.

Although the present disclosure has been described in terms of certain preferred embodiments and illustrations thereof, other embodiments and modifications to preferred embodiments may be possible that are within the principles of the invention. The above descriptions and figures are therefore to be regarded as illustrative and not restrictive.

Thus the scope of the present disclosure is defined by the appended claims and includes both combinations and sub-combinations of the various features described hereinabove as well as variations and modifications thereof, which would occur to persons skilled in the art upon reading the foregoing description.

What is claimed is:

1. A system for monitoring, identifying and reporting impact events in real-time, comprising:
an impact event monitoring device configured to monitor one or more impact events of at least one of: objects; and subjects; through a processing device, whereby the processing device configured to identify one or more accurate positions and locations and sensor data of at least one of: the objects; and the subjects; the processing device configured to enable an image capturing unit to capture and record at least one of: the objects; and the subjects;
a network module configured to report the one or more accurate positions and locations, the sensor data, and one or more media files of the one or more impact events to at least one of: a first computing device; a second computing device; and
an impact event reporting module configured to enable the at least one: the first computing device; and the second computing device; to analyze at least one of:
the one or more accurate positions and locations; the sensor data; and the one or more media files of the one or more impact events; to understand the extent of the one or more impact events.

2. The system of claim 1, wherein the impact event monitoring device comprises one or more first set of sensors configured to measure a linear acceleration, a linear velocity, an angular acceleration, jerks, quaternions, Euler angles, vital statistics, a rotational angle, a geographical location, movement and/or motion and gyroscope vectors of at least one of: the objects; and the subjects.

3. The system of claim 1, wherein the impact event monitoring device comprises one or more second set of sensors configured to calibrate one or more orientations of at least one of: the objects; and the subjects; by measuring Euler angles and/or quaternions.

4. The system of claim 1, wherein the impact event monitoring device comprises one or more third set of sensors configured to monitor one or more vital statistics, rotational angle of a head of the subject at the time of the impact event.

5. The system of claim 4, wherein the one or more third set of sensors are configured to provide an additional sensor data to the second computing device for proper diagnosing extent and severity of the one or more impact events.

6. The system of claim 1, wherein the impact event monitoring device comprises an impact sensing unit configured to detect and determine the one or more impact events that occurs to at least one of: the objects; and the subjects.

7. The system of claim 1, wherein the impact event monitoring device comprises a motion detecting unit configured to measure changes in the one or more orientations for continuous replication of a movement and/or motion of at least one of: the objects; and the subjects.

8. The system of claim 1, wherein the impact event monitoring device comprises a GPS module configured to detect an accurate location of the one or more impact events that occurs to at least one of: the objects; the subjects.

9. The system of claim 1, wherein the network module is configured to establish communication between the impact event monitoring device and at least one of: the first computing device; the second computing device; to deliver one or more notifications of the one or more impact events.

10. The system of claim 9, wherein the network module is configured to send the geographical location as a communication link, and an information identifying the location of at least one of: the objects; and the subjects; to the second computing device for communicating the sensor data stored in a memory unit of the impact event monitoring device.

11. The system of claim 1, wherein the impact event reporting module comprising an event monitoring module configured to read the sensor data of at least one of: the objects; and
the subjects.

12. The system of claim 1, wherein the impact event reporting module comprising an impact analyzing module configured to analyze one or more impact events received from the processing device.

13. The system of claim 1, wherein the image capturing module is configured to move the one or more media files of the one or more impact events front and back with respect to time on the at least one of: the first computing device; and the second computing device.

14. The system of claim 1, wherein the impact event reporting module comprising a position detection module configured to fetch at least one of: at least one of: head positions; and body positions; of the subjects before the one or more impact events and after the one or more impact events.

15. The system of claim 1, wherein the impact event reporting module comprising a location detection module configured to fetch at least one of: a geographical location of the objects; and a geographical location of the subjects.

16. The system of claim 1, wherein the impact event reporting module comprising an alert generating module configured to deliver the one or more notifications of the one or more impact events to the second computing device for estimating the extent of the one or more impact events by a second end user.

17. A system for monitoring, identifying and reporting impact events in real-time, comprising:
an impact event monitoring device configured to monitor one or more impact events of at least one of: objects; and subjects, the impact event monitoring device configured to identify the one or more accurate positions and locations of the at least one of: the objects; and the subjects and activates an impact protocol to establish communication with at least one of: a first computing device; and a second computing device over a network, the impact event monitoring device configured to deliver one or more notifications of the one or more impact events of at least one of: the objects; and subjects to a second computing device over the network.

18. A method for monitoring, identifying and reporting impact events in real-time, comprising:
monitoring at least one of: objects; and subjects; of one or more impact events by an event monitoring device;
detecting one or more accurate positions and locations and sensor data by the impact event monitoring device;
capturing and recording at least one of: the objects; and the subjects;
establishing communication between the impact event monitoring device with at least one of: a first computing device; and a second computing device; through a network module;
reporting at least one of: one or more accurate positions and locations; the sensor data; one or more media files; from the impact event monitoring device to at least one of: the first computing device; the second computing device; and
analyzing at least one of: the one or more accurate positions and locations; the sensor data; the one or more media files of the one or more objects by an impact event reporting module for understanding an extent and severity of the one or more impact events.
